(19) United States
(12) Patent Application Publication
(10) Pub. No.: US 2010/0183725 A1
(43) Pub. Date: Jul. 22, 2010

(54) MULTIPLE ACTIVE PHARMACEUTICAL INGREDIENTS COMBINED IN DISCRETE INHALATION PARTICLES AND FORMULATIONS THEREOF

(75) Inventors: Nahed M. Mohsen, Plymouth, MI (US); Thomas A. Armer, Cupertino, CA (US); Robert O. Cook, Hillsborough, NJ (US)

Correspondence Address: MORRISON & FOERSTER LLP, 755 PAGE MILL RD, PALO ALTO, CA, 94304-1018, US

(73) Assignee: MAP PHARMACEUTICALS, INC., Mountain View, CA (US)

(21) Appl. No.: 11/988,913

(22) PCT Filed: Jul. 17, 2006
(86) PCT No.: PCT/US2006/027977

§ 371 Date: Nov. 23, 2009

Related U.S. Application Data
(60) Provisional application No. 60/699,511, filed on Jul. 15, 2005.

(51) Int. Cl. A61K 9/12; A61K 9/14; A61K 31/58

(52) U.S. Cl. 424/489; 514/171; 424/43; 424/45

(57) ABSTRACT

The present disclosure describe inhalation particles where each discrete unagglomerated inhalation particle comprising 2 or more active pharmaceutical ingredients. In one embodiment, the inhalation particles comprise a first and a second API where the second API covers, at least partially, and protects the first API from degradation or instability. Inhalation particles comprising a first and a second API as described herein have many advantages over present means of delivering two or more APIs. Formulations comprising such inhalation particles are also described.

The present disclosure claims priority to and the benefit of U.S. Provisional Patent Application No. 60/699,511, filed Jul. 15, 2005.

### FIELD OF THE DISCLOSURE

The instant disclosure relates generally to inhalation particles and formulations comprising such particles. The instant disclosure relates specifically to inhalation particles comprising a combination of at least a first active pharmaceutical ingredient and a second active pharmaceutical ingredient where the second active pharmaceutical ingredient functions to protect, at least partially, or modulate the pharmacological availability of the first active pharmaceutical ingredient and formulations comprising such particles. Furthermore, the instant application relates to inhaler devices comprising the inhalation particles and/or formulations described herein. The inhalation particles of the invention are particularly useful in the treatment of respiratory disorders.

### BACKGROUND

The delivery of active pharmaceutical ingredients (APIs) and other therapeutic agents to the respiratory tract via nasal and pulmonary delivery of inhalation particles is widely used for the treatment of a variety of diseases and conditions. Respiratory delivery is accomplished in many ways, such as but not limited to: (i) using an aerosol comprising inhalation particles surrounded by a liquid; (ii) using a multi-dose inhaler; (iii) via the delivery of fine dry powdered inhalation particles via a dry powder inhaler; or (iv) using a nebulizer to nebulize a liquid solution or suspension of the API. The delivery of an API or other therapeutic agents to the respiratory tract offers several advantages, such as, but not limited to, avoidance of metabolism of the drug via the first pass metabolic mechanisms and an increased efficiency of delivery to respiratory tissues (as compared to traditional administration via the bloodstream).

However, delivery of drugs via the respiratory tract is critically dependent on the size of the inhalation particles or droplets containing the inhalation particles or API delivered to the respiratory tract. For efficient delivery to the pulmonary system, inhalation particle in the range of about 0.1 microns to about 10 microns or between about 0.5 microns to about 5.8 microns are required. Inhalation particles of this optimal size range are rarely produced during the crystallization step of the inhalation particles/API, and secondary processes are required to generate inhalation particles in the desired range.

A variety of secondary processes are known for preparing inhalation particles of a desired size range, such as by micronization and nanonization. These methods include mechanical attrition, such as but not limited to, crushing, grinding and milling, and precipitation and/or recrystallization from liquid solutions. For example, U.S. Pat. Nos. 4,107,288, 5,534,270, 6,264,922, 5,429,824 and 6,045,829 (among others) disclose methods for wet and dry milling of drug particles. However, such process can damage the crystalline structure of the drug particles, thereby creating amorphous regions on the surface of the particles. Such amorphous regions may lead to particle instability and/or agglomeration. In addition, such secondary processes involve large thermal and mechanical gradients which can directly degrade the potency and activity of the API, or cause topological imperfections, physical instabilities or chemical instabilities that change, or lead to a change in, the size, shape or chemical composition of the particles on further processing or storage. These secondary processes also impart a substantial amount of free energy to the particles, which is generally stored at the surface of the particles. This free energy stored by the particles produces a cohesive force that causes the particles to agglomerate to reduce this stored free energy. Agglomeration processes can be so extensive that respirable, active particles are no longer present in the formulation and/or can no longer be generated from the formulation due to the high strength of the cohesive interaction. This process is exacerbated in the case of inhalation delivery since the particles must be stored in a form suitable for delivery by an inhalation device. Since the particles are stored for relatively long periods of time, the agglomeration process may increase during storage. The agglomeration of the particles interferes with the re-dispersion of the particles by the inhaler device such that the respirable particles required for pulmonary delivery and/or nasal delivery cannot be generated.

Furthermore, when such methods are used to produce an admixture of two or more APIs by physical blending of inhalation particles of each API, the ratios/consistency of each drug in the produced particle mixture is not easily controlled and is therefore not reproducible. Further the very process of dispersion into an aerosol can partition such admixture blends of particles by impaction or sedimentation based on the effective aerodynamic diameter of each particle or their agglomerate. For example, if the mass median aerodynamic diameter (MMAD) of a particle of API in the blend is only slightly larger than the MMAD of the other particle of API in the blend, then well know aerodynamic effects will partition out the larger particle API, thereby increasing the fraction of the smaller particle API, in the resulting aerosol, causing a shift from the original fixed combination ratio. A difference in MMAD, say 2.0 microns versus 3.0 microns, at flow rates of 60 liters per minute delivered through the upper respiratory tract of a human could theoretically enrich the small particle API content of the aerosol reaching the lung by approximately 25%. Therefore, the ratio of each drug delivered in a given dose is not consistent and may be considerably different than the intended fixed combination ratio. The inconsistency of the dose could cause serious problems especially when an API is delivered in a much higher amount than expected. In addition, for the case of dry powder inhalers formulated with lactose blends, preferential segregation for one API may occur at different particle size fractions from the lactose carrier upon aerosolization yielding reduced aerosol performance and poor dose to dose variation for the product.

An alternative method for the preparation of inhalation particles is by spray-drying a solution of one or several drugs. However, the size and the morphology of the particles obtained are not optimal for pulmonary delivery by inhalation. In addition, certain spray-drying techniques utilize increased temperatures to evaporate solvents used during the particle formation process, which can lead to degradation of the drug(s) contained in the particles. This degradation may be amplified during storage of the inhalation particles. Such degradation leads to chemical inconsistency between doses which can decrease the effectiveness of the drug or lead to serious side effects when delivered to the patient.

The techniques described generally above produce inhalation particles that contain only one API or inhalation particles that contain a combination of APIs where the APIs are commingled with one another as admixtures or physical blends. As a result, certain useful properties of inhalation particles containing one or more APIs cannot be exploited. For example, it would be beneficial when using a combination of APIs to provide an inhalation particle that contained an essentially pure kernel or central unified portion of a first API that is coated or substantially coated with a second API (of course, the first API could also coat or substantially coat a central unified portion of the second API). In this manner certain properties of the inhalation particles could be selected based upon the selection of first and second APIs. Such particles are referred to in the art as core/shell, encapsulated or coated. In one embodiment, the second API could protect the first API from degradation or instability by forming a protective coating around the first API. In such a case a first API that was prone to degradation or instability could be protected from such by the second API. In addition a single, discrete inhalation particle comprising two or more APIs would be advantageous in order to control the delivery of the first and/or second APIs or to control the pharmacological availability of the first and/or second APIs. Such a composition of an inhalation particle and formulations comprising such inhalation particles have not been previously known in the art. Furthermore, a single, discrete inhalation particle comprising two or more APIs would be advantageous since the delivery of both drugs would be directed to a single target cell, maximizing the potential synergy of both APIs and controlling the ratio of delivery of each API to a given cell.

### BRIEF DESCRIPTION OF THE FIGURES

FIG. 1 is a collection of scanning electron microscope images of inhalation particles comprising formoterol fumarate as the first API and budesonide as the second API at high magnifications.

FIG. 2 is a thermogram of the inhalation particles comprising formoterol fumarate as the first API and budesonide as the second API showing the characteristic phase transition temperatures.

FIG. 3 is a graph showing the deposition profile of the neat inhalation particles comprising formoterol fumarate as the first API and budesonide as the second API in a dry powder aerosol test apparatus.

FIG. 4 is a graph showing the mass ratio of the inhalation particles comprising formoterol fumarate as the first API and budesonide as the second API in the various stages of the dry powder aerosol performance test apparatus; the legend T0 indicates no incubation, t& indicates an incubation of 7 days at 25 degrees C./75% relative humidity and T28 indicates an incubation of 28 days at 25 degrees C./75% relative humidity, the numerals 1 and 2 indicate duplicate samples.

FIG. 5 is a graph showing the storage characteristics of a combination particle comprising formoterol fumarate as the first API and budesonide as the second API after freezing of the particles for 28 days and incubation at 40 degrees C./75% relative humidity for 28 days in HFA 134a and HFA 227a; the chemical stability of formoterol fumarate was analyzed on the left-hand side of the graph, while the chemical stability of budesonide was analyzed on the right hand side of the graph.

### DETAILED DESCRIPTION

The present disclosure describes inhalation particles comprising multiple, in one embodiment at least a first and a second, active pharmaceutical ingredients (APIs). The inhalation particles produced are discrete unagglomerated particles, wherein all the APIs in desired ratios are contained with in the discrete particles, and may be prepared by a number of processes known in the art and described herein.

The first and second APIs may be selected as desired. In one embodiment the first and second APIs are selected based on a disease state or condition to be treated. In an alternate embodiment, the first and second APIs are selected based on a chemical characteristic of the first and/or second API. The first or second APIs may be from a number of classes of compounds, such as, but not limited to, small molecules, peptides, polypeptides, proteins, nucleotides, polynucleotides, steroids and the like. Exemplary classes of APIs for use in the present disclosure, include, but not limited to, analgesics, anti-inflammatory agents, anthelmintics, anti-arrhythmic agents, antibiotics (including penicillins), anti-asthma agents, anticoagulants, antidepressants, antidiabetic agents, antiepileptics, antihistamines, antihypertensive agents, antianxiety agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), glucocorticoids, haemostatics, immunological agents, metabolic replacement or supplements, lipid regulating agents, muscle relaxants, parasympathomimetics, parathyroid calcitonin and biphosphonates, prostaglandins, radio-pharmaceuticals, steroids (including sex hormones), anti-allergic agents, sedatives, stimulants and anoretics, sympathomimetics, thyroid agents, vasodilators and xanthines.

The first API and the second API can be independently selected from the above. In certain embodiment, the first and second APIs may be different members within the above classes or other compounds. Furthermore, in certain embodiment, the first and second APIs may be the same API, with the first and second APIs comprising different, salts, polymorphs, isomers, or other modifications. In additional embodiment, the first and second APIs may be the same API or members of the same class with different pharmacological release characteristics.

In one embodiment, the first API is at least as soluble as or less soluble than the second API in a given solvent. In an alternate embodiment, the first API is provided in a solution, suspension or other means by which the first API is made at least as soluble as or less soluble that the second API in a given solvent.

In a particular embodiment of the inhalation particles disclosed, the first API is a bronchodilator agent and the second API is an anti-inflammatory agent. In a further embodiment, the bronchodilator is a beta-agonist and the anti-inflammatory agent is a corticosteroid. As used herein, the term beta agonist includes both short-acting beta agonists (SABAs) and long acting beta agonists (LABAs). The definition of beta agonists is means to be broadly inclusive and is meant to include any compounds classified as beta agonists, whether naturally occurring or synthetically prepared. As used herein, the term “corticosteroid” includes both mineral corticoids and glucocorticoids. The definition of corticosteroid is means to be broadly inclusive and is meant to include any compounds classified as a corticosteroid, whether naturally occurring or synthetically prepared. The beta agonist and corticosteroid APIs may be in pure isomeric forms, mixed isomeric forms, pure polymorphic forms or mixed polymorphic forms. In addition the beta agonist and corticosteroid APIs may be in the form of their hydrates, esters, acetals, salts or other known forms.

Examples of beta agonist APIs include, but are not limited to, salbutamol, formoterol, procaterol, salmeterol, clenbuterol, pirbuterol and the like. Examples of corticosteroid APIs include, but are not limited to, budesonide, dexamethasone, cortisone, prednisone, methylprednisone, hydrocortisone, beclomethasone dipropionate, betamethasone, flunisolide, fluticasone, flumethasone, fludrocortisone, diflorasone diacetate, flunisolide, fluocinolone acetonide, fluocinonide, fluorometholone, flurandrenolide, fluprednisolone, methylprednisone, paramethasone, prednisone, prednisolone, triamcinolone, aclometasone, amcinonide, cortisone, tetrahydrocortisol, clobetasol, ciclesonide, desonide, desiximetasonedeflazacort, halcinonide, medrysone, mometasone, paramethasone, tipredane, triamcinolone, rofleponide, aldosterone, fludrocortisone, and desoxycorticosterone acetate.

In one particular embodiment, the corticosteroid API is budesonide or fluticasone and the beta agonist API is formoterol or salmeterol. Specific embodiments include, but are not limited to, inhalation particles comprising formoterol as the first API and budesonide as the second API, formoterol as the first API and fluticasone as the second API, salmeterol as the first API and budesonide as the second API and salmeterol as the first API and fluticasone as the second API.

The inhalation particles of the present disclosure may be created using methods including, but are not limited to, the use of supercritical fluid (SCF) precipitation or sub-supercritical (i.e., near supercritical) precipitation techniques and solution precipitation techniques. Suitable SCF techniques include, as but not limited to, rapid expansion (RES), solution enhanced diffusion (SEDS), gas-anti solvent (GAS), supercritical antisolvent (SAS), precipitation from gas-saturated solution (PGSS), precipitation with compressed antisolvent (PCA), and aerosol solvent extraction system (ASES). The use of SCF processes to form particles is reviewed in Palakodaty, S., et al., “Phase Behavioral Effects on Particle Formation Processes Using Supercritical Fluids”, Pharmaceutical Research, vol. 16. p. 976 (1999). These methods permit the formation of micron and sub-micron sized particles with differing morphologies depending on the method and parameters selected. Suitable SCF and SEDS processes are also described in WO-95/01221, WO-96/00610, WO-98/36825, WO-99/44733, WO-99/52507, WO-99/52550, WO-99/59710, WO-00/30613, WO-00/67892, WO-01/03821, WO-01/15664, WO-02/058674, WO-02/38127, and WO-03/008082. Furthermore the methods described in U.S. patent application Ser. No. 10/264,030 may be used to prepare such inhalation particles. In addition, the inhalation particles can be fabricated by spray drying, lyophilization, volume exclusion, and any other conventional methods of particle reduction. These methods permit the formation of micron and sub-micron sized particles with differing morphologies depending on the method and parameters selected.

In one particular embodiment, the method used to produce the inhalation particles is a modified ASES system as developed by Eiffel Technologies Limited and as described in a patent application filed on Jul. 15, 2005 and titled “Method of Particle Formation”.

Inhalation particles produced through the use of these methods can be formulated into formulations.

The inhalation particles may be formulated into formulations (such as suspensions) for nebulization by well established methods, such as jet nebulizers, ultrasonic nebulizers, and vibrating orifice nebulizers including Aerogen Aeroneb®, Omron MicroAire®, PARI EFlow™, Boeringher Respimat®, Aradigm AERx®, and next generation nebulizers from Repironics, Ventaira, and Profile Therapeutics. The formulations can be packaged into nebulas by blow/fill/seal technology presented either as a unit container of a biphasic system.

The inhalation particles may also be formulated into aerosol formulations using propellants. Suitable propellants include, but not limited to, hydrofluoroalkanes (HFA) such as the C₁-C₄ hydrofluorocarbons. Suitable HFA propellants, include but are not limited to, 1,1,1,2,3,3,-heptafluoro-n-propane (HFA 227) and/or 1,1,1,2-tetrafluoroethane (HFA 134) or any mixture of both in any proportions. In one embodiment, the mixture of HFA propellants is selected so that the density of the mixture is matched to the density of the inhalation particles in order to minimize settling or creaming of the inhalation particles. Carbon dioxide and alkanes, such as pentane, isopentane, butane, isobutane, propane and ethane, can also be used as propellants or blended with the C₁₋₄ hydrofluroalkane propellants discussed above. The formulation may (but is not required to) further comprise carriers, additives and/or diluents as is known in the art.

The inhalation particles produced may be formulated into dry powder formulations. The particles can be used for pulmonary drug delivery by inhalation directly without added carriers, additives or diluents by packaging the inhalation particles into capsules, cartridges, blister packs or reservoirs of a dry powder inhaler (a variety of dry powder inhalers may be used as is known in the art). The inhalation particles may also comprise one or more carriers, additives or diluents to form loose agglomerates of the inhalation particles that are dispersed into individual inhalation particles by the action of the dry powder inhaler. The formulation may (but is not required to) further comprise carriers, additives and/or diluents as is known in the art. Carriers, alone or in combination with other additives, commonly used include, but are not limited to, lactose, dextran, mannitol and glucose. Carriers may be used simply as bulking agents or to improve the dispersibility of the inhalation particles.

If the formulations comprise a carrier, additive or diluent, the total amount of the APIs is typically about 0.1-99.9% (w/w), about 0.25-75% (w/w), about 0.5-50% (w/w), about 0.75-25% (w/w) or about 1-10% (w/w), based on total weight of the formulation. Such formulations may be prepared by methods known in the art. Formulations as above comprising the inhalation particles described herein may be used for nasal and pulmonary inhalation an appropriate device.

As stated above, the formulations may contain added carriers, additives and diluents. The carriers, additives and diluents can be added in the range of 0.0 to 99.9% (w/w) based on the total weight of the formulation. Additives, include, but are not limited to, stabilizers, excipients preservatives, suspending agents, chelating agents, complexing agents and/or other components known to one or ordinary skill in the art. Such carriers, additives and diluents may be a pharmaceutically acceptable grade. Suitable excipients include, but are not limited to ionic and non-ionic surfactants, polymers, natural products and oligomers. Examples of certain suitable excipients which may be used are disclosed in U.S. Pat. Nos. 6,264,739, 5,145,684, 5,565,188 and 5,587,143. In one embodiment, the excipient is an ionic or non-ionic surfactant. Typical surfactants include, but are not limited to, the oleates, stearates, myristates, alkylethers, alklyarylethers and sorbates and any combination of the foregoing. In a particular embodiment, the surfactant is a polyoxyethylene sorbitan fatty acid ester, such as Tween 20 or Tween 80, sorbitan monooleate (SPAN-80) or isopropyl myristate. Other suitable excipients include polyvinylpyrrolidine, polyethylene glycol, microcrystalline cellulose, cellulose, cellulose acetate, cyclodextrin, hydroxypropyl beta cyclodextrin, lecithin, magnesium stearate, lactose, mannitol, trehalose and the like and any combination of the foregoing. The formulations may also comprise polar solvents in small amounts to aid in the solubilization of the surfactants, when used. Suitable polar compounds include C₂₋₆ alcohols and polyols, such as ethanol, isopropanol, polypropylene glycol and any combination of the foregoing. In the event the inhalation particles are to be formulated for use with a dry powder inhaler, lactose, dextran, mannitol and glucose or other suitable compounds may be used. Suitable preservatives, include, but are not limited to, chlorobutanol and benzalkonium chloride and any combination of the foregoing. Suitable chelating agents include, but are not limited to, EDTA and EGTA and any combination of the foregoing. The formulations described above may comprise additional components as well, such as, but not limited to, suspending agents and other components commonly used and known in the art.

In one embodiment, the inhalation particles comprising a first and a second API as described have a substantially uniform morphology. The inhalation particles comprise a first and a second API in a morphology with respect to one another characterized by the physical positioning of the first and second APIs in the inhalation particle. In one embodiment, the inhalation particles have a fully encapsulated morphology. As used herein, “fully encapsulated” means that the first API is substantially encapsulated within and by the second API. As used herein, “substantially” means the second API covers and/or protects at least 90%, at least 95% or at least 99% of the first API. In such an embodiment, the first API has a surface area exposed at the surface of the inhalation particle of 10% or less of the total exterior surface area of the inhalation particle.

In an alternate embodiment, the inhalation particles have a distributed encapsulated morphology. As used herein, the term “distributed encapsulated” means that the first API is partially encapsulated by the second API. As used herein, “partially” means that certain domains of the first API are completely encapsulated by the second API and certain domains of the first API are exposed on the surface of the inhalation particle. In one example of this embodiment, the first API has a surface area exposed at the surface of the inhalation particle of greater than 10% but less than or equal to 50% of the total exterior surface area of the inhalation particle and the second API covers and/or protects from 89.9% to 50% of the first API. In another example of this embodiment, the first API has a surface area exposed at the surface of the inhalation particle of greater than 10% but less than or equal to 90% of the total exterior surface area of the inhalation particle and the second API covers and/or protects from 89.9% to 10% of the first API. In yet another example of this embodiment, the first API has a surface area exposed at the surface of the inhalation particle of greater than 10% but less than or equal to 99% of the total exterior surface area of the inhalation particle and the second API covers and/or protects from 89.9% to 1% of the first API. In one example of this embodiment, the first API is present in a volume percentage of between 0.1 and 36% by volume.

In a further embodiment, the inhalation particles have a co-continuous matrix morphology (also referred to as a molecular dispersion, or interpenetrating network). As used herein, the term “co-continuous matrix” means the first and second APIs have an equal or substantially equal surface area exposed on the surface of the inhalation particle. As used herein, the term substantially equal means within 10% (v/v). In one embodiment where the inhalation particles have a co-continuous matrix morphology, the first API is present at a concentration (w/w) equal to, less than or greater than the second API.

It will be understood that the above examples of morphology describing the inhalation particles are not all inclusive and should not be taken as limiting the composition or structure of the inhalation particles described herein. Furthermore, a plurality of inhalation particles or an formulation comprising a plurality of inhalation particles as described herein may have any combination of morphologies as described above. However, in one embodiment at least 50%, 60%, 70%, 80%, 90%, 95% or 99% of the inhalation particles have a single morphology. In a specific embodiment, the single morphology is selected from the group consisting of fully encapsulated morphology, dispersed encapsulated morphology and co-continuous matrix morphology.

Regardless of morphology of the inhalation particles, the presence of the first and the second API in each discrete inhalation particle promotes the coincidental delivery of the first and second API. As used herein, the term “coincidental delivery” means that the first and second APIs are delivered to the same cell at the same time. The coincidental delivery of the first and second APIs offers therapeutic advantages not previously known in the art. The two leading mechanisms to explain this therapeutic advantage are (1) activation or ‘priming’ of the glucocorticoid receptor (GR) by the beta-agonists making it more receptive to the inhaled corticosteroid and (2) the increased translocation of the inhaled corticosteroid-glucocorticoid receptor complex into the cell nucleus (where the complex exerts biological activity) by the beta-agonists. For example, when two or more drugs are formulated together such that each drug is present in discrete particles, the delivery of each drug to the same cell and/or the order of delivery cannot be controlled. Therefore, it is impossible to ensure that each cell in need of treatment receives each drug. The inhalation particles of the present disclosure solve this problem. Furthermore, by selecting the desired morphology and the first and second API, not only can the coincidental delivery of the first and second APIs be ensured, the order of release of the first and second APIs can be controlled and determined to achieve maximum therapeutic benefit.

It is well known in the art that the size of an inhalation particle determines the depth of penetration into the lung. The depth of penetration is important for achieving the desired therapeutic benefit. In one embodiment, the inhalation particles have a particle size (i.e., MMAD) less than about 10 microns in diameter, less than about 7.0 microns in diameter, less than about 5.8 microns in diameter, less than about 3 microns in diameter or less than about 1.5 microns in diameter. In certain embodiments, at least 80%, at least 90% or at least 95% of the total inhalation particles in a given formulation have an average particle size less than 7.0 microns in diameter. In further embodiments, at least 80%, at least 90% or at least 95% of the total inhalation particles in a given formulation have an average particle size less than 5.8 microns in diameter. In one embodiment, the inhalation particles have a particle size greater than about 0.1 microns in diameter, greater than about 1.0 microns in diameter, or greater than about 1.2 microns in diameter. In certain embodiments, at least 80%, at least 90% or at least 95% of the total inhalation particles in a given formulation have an average particle size greater than 0.1 microns in diameter. In further embodiments, at least 80%, at least 90% or at least 95% of the total inhalation particles in a given formulation have an average particle size less than 1.2 microns in diameter.

Each of the particles has a predetermined and constant mass ratio of the first and second APIs with respect to one another. By constant, it is meant that at least 80%, 90%, 95%, 99% or greater (by mass) of the inhalation particles have the predetermined mass ratio of the first to the second API. For example, the predetermined mass ratio is 1:18 to 1:36 (first API to second API), the mass ratio is constant equal if 80% or greater of the particles have a ratio of first to second API in the range of 1:18 to 1:36.

The particle size may be determined by means known and standard in the art such as a cascade impactor, such as an Anderson Cascade Impactor also known as an “Apparatus 1” per USP 601. It is generally known that stages 3-6 detect inhalation particles having a size between about 1.2 and 6.5 microns and that stages 3-8 detect inhalation particles having a size between about 0.26 and 6.5 microns. Inhalation particle sizes between about 1.2 and 6.5 microns or between about 0.26 and 6.5 microns are known as the effective particle size range or the fine particle fraction. In one embodiment, all the inhalation particles have a predetermined and constant mass ratio across the fine particle fraction. In a further embodiment, the entire range of inhalation particle sizes have, a predetermined and constant mass ratio.

The mass ratio of the first API to the second API can be varied and may depend on the chemical identity of the first and second APIs, the application of the inhalation particles containing the first and second APIs and method used to produce the inhalation particles containing the first and second APIs. In one embodiment the mass ratio of the first to second API ranges from 50:1 to 1:500. In another embodiment, the mass ratio of the first to second API is from 1:5 to 1:500. In a further embodiment the mass ratio of the first to second API ranges from 1:1 to 1:250. In still another embodiment the mass ratio of the first to second API ranges from 1:1 to 1:80. In another embodiment, the mass ratio of the first to second API ranges from 1:18 to 1:36. In yet another embodiment, the mass ratio of the first to second API is 1:20. When the first API is a beta agonist and the second API is a corticosteroid, the mass ratio of the first API to the second API may be selected from the ranges given above.

The present disclosure describes inhalation particles, and formulations comprising such particles, comprising two or more APIs where at least about 80%, 90% or 95% of the inhalation particles have a size range of 1.2 to 6.5 microns, with 80%, 90% or 95% of said inhalation particles in the 1.2 to 6.5 micron size range having a predetermined and constant mass ratio of first and second (or additional) APIs, with said particles having a substantially uniform appearance and morphology capable coincidental delivery of the first and second (or additional) APIs. The inhalation particles so described are especially useful for inhalation delivery by dry powder inhalers, metered dose inhalers and/or nebulizers.

All patents, patent applications and publications referred to herein are incorporated by reference to the extent fully set forth herein. Reference to any of the above mentioned materials is not an acknowledgement that these materials would be recognized to teach or suggest or be regarded as relevant by those of ordinary skill in the art.

### EXAMPLES

### Example 1

Inhalation particles comprising formoterol fumarate as the first API and budesonide as the second API were produced using a modified ASES system as developed by Eiffel Technologies Limited and as described in Australian Patent Application filed on Jul. 15, 2005 and titled “Method of Particle Formation”. The resultant inhalation particles had a formoterol to budesonide mass ratio of 1:20.

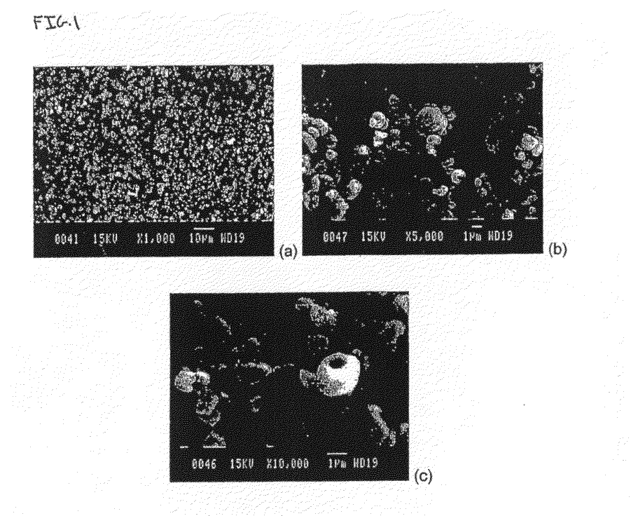

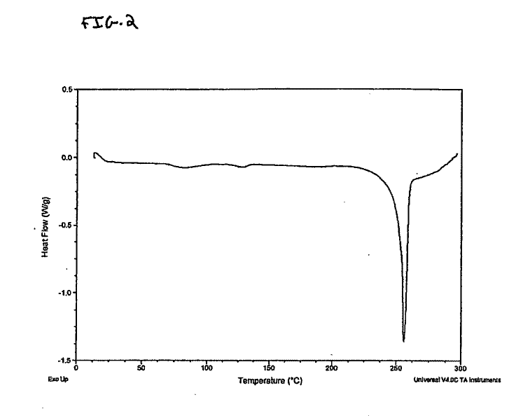

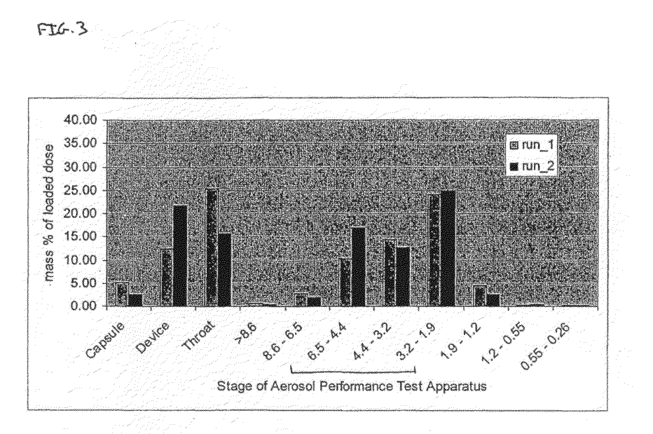

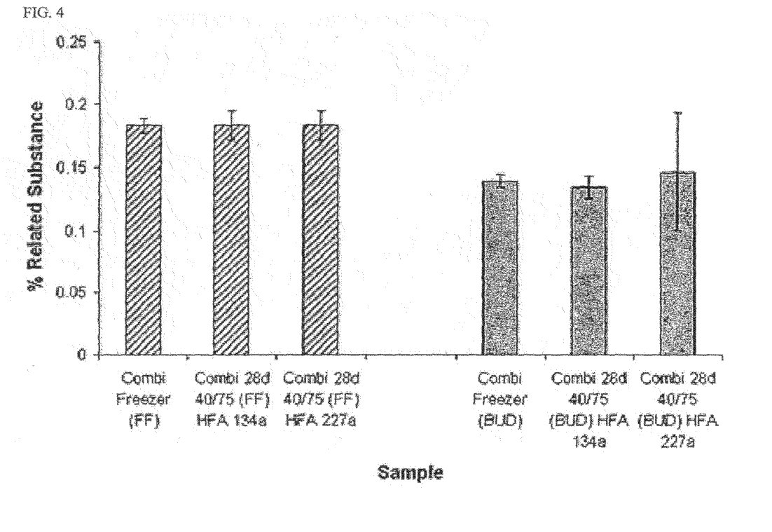

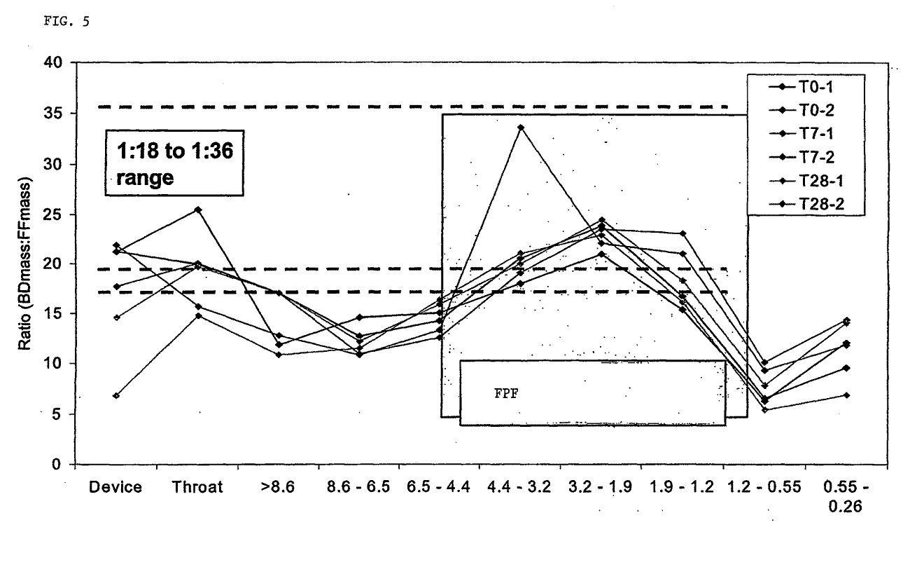

The physical and thermal characteristics of the formoterol/budesonide inhalation particles are shown in FIGS. 1-4 and in Table 2. The inhalation particles were in the form of unagglomerated, discrete, fine, white, easily-dispersible powder consisting of mainly toroidal-shaped particles of less than 5 micron in diameter when viewed under SEM (FIG. 1). The inhalation particles had a major single endothermic peak at approximately 256.0° C. and two phase transition points at approximately 82.5 and 127.8° C. which resembled the thermal changes observed for crystalline formoterol (FIG. 2). In an Aerosizer device tested with an Anderson Cascade Impactor with pre-separator and eight stages (refer to Table 1 for the parameters used), the inhalation particles, in their dry powder, neat form had an average emitted dose of 79.2% by mass, an average fine particle fraction of 70.6% by mass (as a percentage of the emitted dose), and an average fine particle fraction of 55.8% by mass (as a percentage of the loaded dose). These performance figures are shown in Table 2. Corresponding device deposition profiles of the combination product are shown in FIG. 3. At least 95% by mass of the fine particle fraction deposited on stages 3-6 inclusively, which corresponds to the approximate particle size range 1.2-6.5 micron, and that on each of these stages the formoterol to budesonide mass ratio of the individual inhalation particles was the target ratio of about 1:20. The budesonide to formoterol mass ratio in each of the stages of the aerosol performance test device is shown in FIG. 4. FIG. 5 demonstrates that the combination particles described herein are stable under a wide variety of storage conditions. Particles comprising formoterol fumarate as the first API and budesonide as the second API were assayed for chemical stability after freezing of the particles for 28 days and incubation of the particles at 40 degrees C/75% relative humidity for 28 days in HFA 134a and HFA 227a. The chemical stability of formoterol fumarate was analyzed on the left-hand side of the graph, while the chemical stability of budesonide was analyzed on the right hand side of the graph.

The actual mass ratio of the total combined fine particle fractions (from Stages 3 to 8 inclusive) was calculated to be 1:19. However, the actual mass ratio for Stages 3 to 8 is likely to be higher (i.e. closer to 1:20) than the calculated mass ratio of the inhalation particles recovered from Stages 1 (>8.6 pm), 7 (1.2-0.55 pm) and 8 (0.55-0.26 pm) because the powder at these Stages may have been underestimated due to the low amounts of inhalation particles collected on those Stages (see FIG. 3). This is because the amount of formoterol fumarate present was close to the limit of quantitation for the formoterol assay.

The inhalation particles produced as described have a fully coated and/or a distributed encapsulated morphology. In this manner the budesonide (second API) coats and protects the formoterol fumarate (first API) from degradation and instability that is characteristic of formoterol inhalation particles. Therefore, the formoterol/budesonide inhalation particles described herein show greater stability than the inhalation particles known heretofore in the art. A list of definitions and analytical derivations used in the performance indicators for the dry powder aerosol performance tests is given in Table 3.

### Example 2

Inhalation particles comprising salmeterol xinofoate as the first API and fluticasone propionate as the second were prepared using the method described in Example 1. All other parameters were the same as in Example 1 above. The inhalation particles were less than 5 micron diameter and was characterized by a clear endothermic event occurring at approximately 266° C. on a DSC thermogram.

### Example 3

Inhalation particles comprising formoterol fumarate as the first API and fluticasone propionate as the second were prepared using the method described in Example 1. All other parameters were the same as in Example 1 above. The inhalation particles were less than 5 micron diameter and was characterized by a clear endothermic event occurring at approximately 266° C. on a DSC thermogram.

### Example 4

Inhalation particles comprising salmeterol xinofoate as the first API and budesonide as the second were prepared using the method described in Example 1. All other parameters were the same as in Example 1 above. The inhalation particles were less than 5 micron diameter and was characterized by a clear endothermic event occurring at approximately 256° C. on a DSC thermogram.

### Example 5

The following examples illustrated in Table 4 are prophetic examples of selected embodiments aerosol formulations capable of being produced using the inhalation particles described. The embodiments are not to be interpreted as limiting in any way but merely indicative of the aerosol formulations capable of being produced using the inhalation particles described herein. The aerosol formulations may be prepared as would be known to one of ordinary skill in the art. Exemplary methods for formulation are given in U.S. patent application Ser. No. 10/264,030.

TABLE 1
A list of the method parameters used in
the dry powder aerosol performance tests.

| | |
|---|---|
| Dry powder device | Aerolizer (Novartis, fitted snout) |
| Loaded dose, mg | 10 ± 1 |
| Capsule type | Gelatin, size no. 3 |
| Cascade impaction device | Anderson cascade impactor with |
| | pre-separator and 8 stages |
| Air flowrate, L/min | 60 |
| Coating on plates | Propylene glycol |
| Filter type | Glass fibre |
| Actuation period, s | 60 |
| No. of actuations per run | 1 |
| No. of replicates | 2 |
| Wash solvent | Methanol (technical grade) |

TABLE 2
A list of the aerosol performance indices and data of
the neat formoterol/budesonide combination product in
the dry powder aerosol performance test apparatus.

| Performance Index | Run 1 | Run 2 | Average |
|---|---|---|---|
| % ED | 82.5 | 75.9 | 79.2 |
| % FPF of ED | 65.2 | 76.0 | 70.6 |
| % FPF of LD | 53.8 | 57.7 | 55.8 |

TABLE 3
A list of the definitions and analytical derivations
used as the performance indicators for the
dry powder aerosol performance tests.

| | |
|---|---|
| Loaded dose, LD | Total mass recovered |
| Emitted dose, ED | Total mass recovered in the Throat and 8 |
| | collection plates of the cascade impactor |
| | apparatus |
| Fine particle fraction, FPF | Total mass recovered from the collection |
| | plates 3 to 6 |
| % ED | ED/LD × 100 |
| % FPF of ED | FPF/ED × 100 |
| % FPF of LD | FPF/LD × 100 |

TABLE 4
Exemplary Aerosol Formulations

| | Inhalation Particles | | | | | |
|---|---|---|---|---|---|---|
| | Comprising First and | | | Polar | | |
| | Second API* | Surfactant 1 | Surfactant 2 | Compound | p134a | p227 |
| | (milligrams) | (milligrams) | (milligrams) | (milligrams) | (grams) | (grams) |
| 1 | 50.0 | 0.0 | 0.0 | 0.0 | 0.0 | 12.00 |
| 2 | 50.0 | 0.0 | 0.0 | 0.0 | 12.0 | 0.0 |
| 3 | 50.0 | 0.0 | 0.0 | 0.0 | 6.0 | 6.0 |
| 4 | 50.0 | 0.0 | 0.0 | 0.0 | 8.4 | 3.6 |
| 5 | 50.0 | 0.0 | 0.0 | 0.0 | 3.6 | 8.4 |
| 6 | 50.0 | 0.5 | 0.0 | 0.0 | 12.0 | 0.00 |
| 7 | 50.0 | 0.5 | 0.0 | 0.0 | 0.0 | 12.00 |
| 8 | 50.0 | 0.5 | 0.0 | 0.0 | 6.0 | 6.0 |
| 9 | 50.0 | 0.0 | 1.0 | 0.0 | 12.0 | 0.00 |
| 10 | 50.0 | 0.0 | 1.0 | 0.0 | 0.0 | 12.00 |
| 11 | 50.0 | 0.0 | 1.0 | 0.0 | 6.0 | 6.0 |
| 12 | 50.0 | 0.0 | 0.0 | 0.1 | 0.0 | 12.00 |
| 13 | 50.0 | 0.0 | 0.0 | 0.1 | 12.0 | 0.0 |
| 14 | 50.0 | 0.0 | 0.0 | 0.1 | 6.0 | 6.0 |
| 15 | 50.0 | 0.5 | 1.0 | 0.1 | 0.0 | 12.00 |
| 16 | 50.0 | 0.5 | 1.0 | 0.1 | 12.0 | 0.0 |
| 17 | 50.0 | 0.5 | 1.0 | 0.1 | 6.0 | 6.0 |
| 18 | 50.0 | 1.0 | 1.0 | 0.1 | 0.0 | 12.00 |
| 19 | 50.0 | 1.0 | 1.0 | 0.1 | 12.0 | 0.0 |
| 20 | 50.0 | 1.0 | 1.0 | 0.1 | 6.0 | 6.0 |

*The amount of first and second API is given for exemplary purposes only. The amount of first and second API may be varied, such as, but not limited to, from 1 to 200 mg. The amounts of Surfactant 1, Surfactant 2, Polar Compound, p134a and p227 may also be varied if desired when the amounts of the first and second API are varied.

1. A plurality of inhalation particles, each of said particles comprising at least a first second active pharmaceutical ingredient (API) and a second API, the first and second API have a defined morphology with respect to one another within said particle and the first API being in a predetermined and constant mass ratio with regard to the second API.

2. The particles of claim 1 where the morphology is a fully encapsulated morphology.

3. The particles of claim 2 where the first API has a surface area exposed on the surface of the particle of 10% or less of the total exterior surface area of the particle.

4. The particles of claim 2 where the second API covers or protects at least 90% of the first API.

5. The particles of claim 2 where the first API has a surface area exposed on the surface of the particle of 1% or less of the total exterior surface area of the particle.

6. The particles of claim 2 where the second API covers or protects at least 99% of the first API.

7. The particles of claim 2 where the first API is present in a mass ratio to the second API of from 5:1 to 1:100.

8. The particles of claim 2 where the first API is present in a mass ratio to the second API of from 1:18 to 1:36.

9. The particles of claim 1 where the morphology is a distributed encapsulated morphology.

10. The particle of claim 9 where the first API has a surface area exposed on the surface of the particle of greater than 10% but less than or equal to 50% of the total exterior surface area of the particle.

11. The particle of claim 9 where the second API covers and/or protects from 89.9% to 50% of the first API.

12. The particles of claim 9 where the first API has a surface area exposed on the surface of the particle of greater than 10% but less than or equal to 90% of the total exterior surface area of the particle.

13. The particles of claim 9 where the second API covers and/or protects from 89.9% to 10% of the first API.

14. The particles of claim 9 where the first API has a surface area exposed on the surface of the particle of greater than 10% but less than or equal to 99% of the total exterior surface area of the particle.

15. The particles of claim 9 where the second API covers and/or protects from 89.9% to 1% of the first API.

16. The particles of claim 9 where the first API is present in a mass ratio to the second API of from 5:1 to 1:100.

17. The particles of claim 9 where the first API is present in a mass ratio to the second API of from 1:18 to 1:36.

18. The particles of claim 1 where the morphology is a co-continuous matrix morphology.

19. The particles of claim 18 where the first API has a surface area exposed on the surface of the particle of from about 40% to about 60%.

20. The particles of claim 18 where the second API covers and/or protects from about 40% to about 60% of the first API.

21. The particles of claim 18 where the first API is present in a mass ratio to the second API of from 5:1 to 1:100.

22. The particles of claim 18 where the first API is present in a mass ratio to the second API of from 1:18 to 1:36.

23. The particles of claim 1 where said first API is equally soluble or less soluble than the second API in a given solvent.

24. The particles of claim 1 where said particle has a uniform shape.

25. The particles of claim 1 where said particle has a toroidal shape.

26. The particles of claim 1 where said particles have a particle size of less than or equal to 10 microns in diameter, less than or equal to 7 microns in diameter, less than or equal to 5.8 microns in diameter, less than or equal to 3 microns in diameter, or less than or equal to 1.5 microns in diameter.

27. The particles of claim 1 where at least 90% of the particles have a particle size greater than 0.1 microns in diameter and less than 10 microns in diameter.

28. The particles of claim 1 where at least 90% of the particles have a particle size greater than 0.1 microns in diameter and less than 5.8 microns in diameter.

29. A plurality of inhalation particles, each of said particles comprising formoterol fumarate as a first API and budesonide as a second API, the first and the second API being in a fully encapsulated morphology with respect to one another within said particle and said first API being in a predetermined and constant mass ratio.

30. The particles of claim 29 where said ratio is from about 1:18 to about 1:36.

31. The particles of claim 29 where said ratio is about 1:20.

32. The particles of claim 29 where said particles have a uniform shape.

33. The particles of claim 29 where said, particles have a toroidal shape.

34. A plurality of inhalation particles, each of said particles comprising formoterol fumarate as a first API and budesonide as a second API, the first and the second API being in a partially encapsulated morphology with respect to one another within said particle and said first API being in a predetermined and constant mass ratio.

35. The particles of claim 34 where said ratio is from about 1:18 to about 1:36.

36. The particles of claim 34 where said ratio is about 1:20.

37. The particles of claim 34 where said particles have a uniform shape.

38. The particles of claim 34 where said particles have a toroidal shape.

39. A formulation for inhalation comprising a plurality of particles, each of said particles comprising at least a first second active pharmaceutical ingredient (API) and a second API, the first and second API have a defined morphology with respect to one another within said particle, and the first API being in a predetermined and constant mass ratio with regard to the second API.

40. The formulation of claim 39 where the first API is formoterol fumarate and the second API is budesonide.

41. The formulation of claim 39 where said formulation is an aerosol formulation comprising one or more propellants.

42. The formulation of claim 41 where the first API is formoterol fumarate and the second API is budesonide.

43. The formulation of claim 41 where at least 90% of the particles have a particle size greater than 0.1 microns in diameter and less than 10 microns in diameter.

44. The formulation of claim 41 where at least 90% of the particles have a particle size greater than 0.1 microns in diameter and less than 5.8 microns in diameter.

45. The formulation of claim 41 where the first API is equally soluble or less soluble than the second API in a given solvent.

46. The formulation of claim 41 where each of said particles has a uniform shape.

47. The formulation of claim 41 where each of said particles has a toroidal shape.

48. The formulation of claim 41 where the propellant is a C₁-C₄ hydrofluorocarbon propellant.

49. The formulation of claim 41 where the propellant is 1,1,1,2,3,3,-heptafluoro-n-propane (HFA 227) and/or 1,1,1,2-tetrafluoroethane (HFA 134) or any mixture of both in any proportions.

50. The formulation of claim 41 further comprising a carrier, a stabilizer, an excipient, a preservative, a suspending agent, a chelating agent, a complexing agent, a diluent, a co-solvent or a combination of any of the foregoing.

51. The formulation of claim 39 where said formulation is a dry powder formulation.

52. The formulation of claim 51 where the first API is formoterol fumarate and the second API is budesonide.

53. The formulation of claim 51 where at least 90% of the particles have a particle size greater than 0.1 microns in diameter and less than 10 microns in diameter.

54. The formulation of claim 51 where at least 90% of the particles have a particle size greater than 0.1 microns in diameter and less than 5.8 microns in diameter.

55. The formulation of claim 51 where the first API is equally soluble or less soluble than the second API, and the first API and

56. The formulation of claim 51 where each of said particles has a uniform shape.

57. The formulation of claim 51 where each of said particles has a toroidal shape.

58. The formulation of claim 51 further comprising a carrier selected from the group consisting of lactose, dextran, mannitol and glucose.

59. The formulation of claim 39 where said formulation is a nebulizer formulation.

60. The formulation of claim 59 where the first API is formoterol fumarate and the second API is budesonide.

61. The formulation of claim 59 where at least 90% of the particles have a particle size greater than 0.1 microns in diameter and less than 10 microns in diameter.

62. The formulation of claim 59 where at least 90% of the particles have a particle size greater than 0.1 microns in diameter and less than 5.8 microns in diameter.

63. The formulation of claim 59 where the first API is equally soluble or less soluble than the second API, and the first API and

64. The formulation of claim 59 where each of said particles has a uniform shape.

65. The formulation of claim 59 where each of said particles has a toroidal shape.

66. An inhaler device comprising a formulation of claim 39.

67. The device of claim 66 where the device is a jet nebulizer, an ultrasonic nebulizer, a vibrating orifice nebulizer, a dry powder inhaler or a metered dose inhaler.

68. An aerosol can containing a formulation of claim 39.
